(19) United States
(12) Patent Application Publication
(10) Pub. No.: US 2005/0196468 A1
(43) Pub. Date: Sep. 8, 2005

(54) Composition and method for nutritional supplement for drug abuse treatment and rehabilitation

(76) Inventor: Ajibike Omosalewa Salako, Laurel, MD (US)

Correspondence Address: LAW OFFICES OF ROYAL W. CRAIG;A PROESSIONAL CORPORATION, SUITE 153, 10 NORTH CALVERT STREET, BALTIMORE, MD, 21202, US

(21) Appl. No.: 11/009,948

(22) Filed: Dec. 10, 2004

Related U.S. Application Data
(60) Provisional application No. 60/528,755, filed on Dec. 10, 2003.

(51) Int. Cl. A61K 33/06; A61K 31/4415; A61K 31/198

(52) U.S. Cl. 424/682; 514/561; 514/563; 514/567; 514/350

(57) ABSTRACT

A cost-effective multi-drug nutritional treatment approach in combination form comprising a specific protocol of specific nutritional supplements for different types of addictions within the distinct phases of recovery, i.e., detoxification, craving and maintenance. The protocol provides the combination of nutritional supplements, dosage and length of treatment. The compositions of the present invention are obtained by combining Bio-Amino caps, Biovitamins, Ca & Mg, Vitamin B6 and Bio-GLA for all treatments and one of L-Glutamine, Tyrosine, Endorphenyl and stronger dosages of Bio-GLA for specific drug addictions, the dosages varying amongst the distinct phases of recovery, i.e., detoxification, craving and maintenance.

### CROSS-REFERENCE TO RELATED APPLICATIONS

The present application derives priority from U.S. Provisional Application 60/528,755 filed Dec. 10, 2003.

### BACKGROUND OF THE INVENTION

1. Field of the Invention

The present invention relates generally to a composition and method for nutritional supplement, and more particularly to a nutritional supplement and methods of use thereof for improved drug abuse detoxification, medical craving management and rehabilitation.

2. Description of the Background

Substance abuse is the non-prescription use of drug(s), which may be licit or illicit. The abusive use has several manifestations, such as 1) use in the strength and dosage prescribed, but used for an unintended purpose; 2) use to prevent or cure a disease; and 3) use to alter the psychological state. All of these abusive uses result in harm to the individual user. In many cases, the abusive use alters the psychological state of the individual, causing addiction and dependency on the drug whereby the individual requires the continued presence of the drug to function normally. Liska, “Drug and the human body: with implications for society” NY: Macmillan, 1986. Addictive drugs strongly activate the brain's reward mechanisms, chemically alter the normal function of these systems, and cause addiction. The promise of reward/pleasure is intense, causing the user to crave the drug and to focus his or her activities around taking the drug. These drugs are referred to as psychoactive because they can cross the blood brain barrier. They affect either the excitatory or inhibitory neurotransmitters. Neurotransmitters are naturally occurring bio-chemicals that transmit information from one neuron to another in the brain, and underlie every thought and emotion, memory and learning. They receive messages, process the messages, and send out the results as new messages to other cells. They are secreted by neurons, the microscopic units of the nervous system, which is comprised of the brain and spinal cord. Neurons have specialized cells called “receptors”. Neurotransmitters recognize specific receptors and grab onto them: a process called “binding”. Receptors bind neuro-transmitters before they can transmit information from one neuron to another. Binding causes a set of chemical reactions within the receiving neuron, which start up the same kind of impulse that was fired in the sending neuron. The result is a change in thinking, feeling or behavior. After binding, receptors let go of the neurotransmitters. Once the receptor lets go, enzymes can either destroy the neurotransmitter or proteins can transport it back to the neuron from which it came. This process is called re-uptake, which allows the neurotransmitters to be recycled, i.e., used over again. Substances of abuse, when present in the brain, can similarly bind these receptors and displace the neuro-transmitters. Substances of abuse that affect the excitatory neuro-transmitters are often referred to as “uppers”, while substances of abuse that affect the inhibitory neuro-transmitters are often referred to as “downers.”

Substances of abuse are classified into four major groups as shown below. Drugs in each group are similar pharmacologically and produce a similar withdrawal syndrome.

Stimulants (Uppers)—e.g., caffeine, nicotine, amphetamines, Met amphetamines, cocaine

Depressants (Downers)—e.g., alcohol, barbiturates, tranquilizers. solvents/glue

Opioids—e.g., Opium, morphine, codeine, heroin

Hallucinogens—e.g., LSD, PCP, Marijuana

Continued exposure to addictive drugs induces adaptive changes in the user's brain cells and neural functioning. The changes vary depending upon the drug of abuse, but can create several pharmacological and physiological effects, such as euphoria (sometimes referred to as a “high”), tolerance (cellular adaptation that leads to increased use, craving, and dependence. The effect of tolerance is that the user needs increasing amounts of the drug to achieve the same effect. The effect of physical drug dependence is that the user's brain cells require the drug to function. Physical drug dependence can be mild, moderate or severe. When a dependent individual becomes preoccupied with seeking and using drugs, the dependency becomes addiction. Other detrimental effects of substance abuse are neuro-biochemical imbalance and anatomical damages. Some commonly abused drugs, such as alcohol, cocaine, marijuana, heroin, morphine, amphetamines, LSD, PCP, barbiturates, solvents, glue and Valium (tm) generate a feeling of euphoria (of being “high”), analgesia, hallucination, sedation, or depression in the abusing user following consumption of the drug.

The World Health Organization considers cocaine the most reinforcing, and therefore the most addicting drug. Thus, the feeling of euphoria (being “high”), as a pharmacological effect can best be explained in terms of cocaine use. Kuhar suggests a “dopamine hypothesis” for the euphoric (reinforcing properties of cocaine. Kuhar M J, Ritz M C, Boja W, “The dopamine hypothesis of the reinforcing properties of cocaine”, Trends in Neuro-science (1991) 14(7): 299-302. A common factor in mood-altering drugs is their ability to elevate the levels of the substance in the brain called dopamine. Dopamine is a neurotransmitter. The surge of dopamine in a drug addict's brain triggers cocaine high, for example. While serotonin, another neurotransmitter, is associated with feelings of sadness, dopamine is associated with pleasure and elation. The essence of this dopamine hypothesis is that cocaine binds at the dopamine receptor and mainly inhibits neuro-transmitter re-uptake; the resulting potentiation of doperminergic neuro-transmission in the mesolimbocortical pathways ultimately causes euphoria.

Craving occurs whenever the drug of abuse has been metabolized and is no longer present at the receptor site, or when the neurotransmitter is not present. In his book, Drugs and Life, Avis discusses the mechanism of craving. According to Avis, whenever a neurotransmitter is released or a drug that mimics a neurotransmitter is transported to the receptors, it must be bound for a neural message to be transmitted. When neither the drug of addiction not the neurotransmitter is bound, craving occurs. Avis H., “Drugs and Life”, Dubuque, Iowa, Wm. C. Brown (1990), pp 1-50. Addicts therefore do not crave cocaine per se, but rather the rush of dopamine that the drug produces.

The area of the brain affected by addictive drugs is called the limbic system, which is a complex group of nuclei and tracts present in the mid-brain. The limbic system includes the hypothalamus (responsible for memory and drive for hunger, sex, and aggression), thalamus (serves as a relay center for pain sensations), amygdale (responsible for motivation and aggression), and part of cerebral cortex (responsible for motor coordination). The system integrates emotional and motivational behavior, particularly motor coordination in emotional responses. Rathus S A, “Psychology: Annotated Instructor's Edition” 4^{th} Ed, Forth Worth: Holt, Reinhart and Winston (1990). It is therefore logical to expect that nutritional supplements may be useful in replenishing what the brain is missing as a result of these illicit agents.

Until recently, the conventional treatment of three-day detoxification (which includes the use of Valium (tm) and barbiturates), counseling, twelve step program and psychiatric treatment has been the most popular approach in the management of substance abuse. However, these approaches have met with limited success, particularly in the areas of relapse and harm reduction, because they have not addressed the neuro-biochemical imbalance of these substances. Julia Ross, Salako-Akande, “Nutritional Supplements: As Adjunct To Effective Management Of Craving And Withdrawal In The Drug Addicted” US Nigerian Voice (1996). Conventional long-term treatment after detoxification has addressed only the psychological aspect of craving and not its biochemical (medical) aspect.

Some of the aforementioned illicit drugs have similar biochemical structures to neurotransmitters, and may displace them, making them unavailable to the brain. Illicit drugs may also deceive the brain to recognize them as neurotransmitters or to act on the satiety center to make the user feel adequately fed with food, which leads to the user lacking nutrients that otherwise come from food. Giardano D., “Drug Education: Content and Methods”, 4^{th} Ed. Newberry Award Records (1988) pages 1-71. Thus, long-term recovery is usually impossible unless the missing neuro-chemicals or the nutrients needed to produce them are provided. Blum K., Neuro-Nutrition As An Adjunct To Therapy For Addictive Disease”, The Nutrition Report 198: 7(6); 41-46.

Conventional methods of detoxification use addictive chemicals and more relapses occur because of continuous and persistent craving. These conventional methods include the use of Valium (tm) and barbiturates in the detoxification of alcohol and heroin. Methadone is also used as a replacement therapy for heroin. Because Valium (tm) and barbiturates are also addictive drugs, they can create tolerance, which is cellular adaptation with eventual need to increase use. Thus, there is a serious potential risk of overdose. The combined medication treatment can be very expensive. During detoxification, the subject goes through withdrawal and feels a significant degree of discomfort, which results from parasympathetic outflow from the brain, which has been disrupted by the non-availability of the neurotransmitter, displacement, and/or presence of drug of abuse, which is foreign to the brain. The rate of recidivism (return to treatment) is consequently high. Conventional treatment can be very expensive and unrewarding.

Current studies of complementary and alternative treatments show that nutritional supplements are able to significantly relieve craving, depression and exhaustion, which lead addicts to relapse following detoxification. Julia Ross, Salako-Akande, Supra.

Nutritional supplements are basic amino acids and proteins that act as building blocks for the body cells, tissues and chemical substances. They also include vitamins and minerals that may act as facilitators for body building. The brain, as a part of the body, needs these supplements to produce the neurotransmitters, which are needed for basic bodily functions and emotional responses. Nutritional supplements can be useful in replenishing the chemicals that the brain is missing as a result of the addictive drug.

Several studies have shown the effectiveness of nutritional supplements in the management of substance abuse. For example, Blum, K. Neuro-nutrition as an Adjunct to Therapy for Addictive Disease. The Nutrition Report 198: 7(6); 41-46, and Mathews-Larson, J. Alcoholism. The Biochemical Connection. NY, Vilard Books. 1992; 103-105 are two such studies. In other studies, neuro-nutrition has been used as an adjunct to therapy for addictive disease. Specifically, massive nicotinic acid therapy of alcoholics is now well known and has previously been shown in experimental animals. Fincle. Louise P, “Experiments in Treating Alcoholics with Glutamic Acid and Glutamine”, Symposium, Biochemical And Nutritional Aspects Of Alcoholism, NY (1964) pp26-37. See also, Smith, Russel F., “Five Year Field Trial Of Massive Nicotinic Therapy Of Alcoholics In Michigan”, Journal Of Orthomolecular Psychiatry (1974). Also, Elson M. Haas, M. D., in his Nutritional Program for Drug Detoxification, excerpted from Staying Healthy with Nutrition: The Complete Guide to Diet and Nutritional Medicine, provides a general nutritional program for drug detoxification and for support during drug use. His nutrient program includes a range of many nutrients, but not the composition or regimen of the present invention.

The present invention adopts the use of nutritional supplements in detoxification as an alternative to the traditional use of addictive tranquilizers and hypnotics. The current method is a cost-effective multi-drug nutritional treatment approach in combination form. The inventor's studies have shown that the supplements can improve neural conduction in those subjects with severe addiction (particularly addition to heroin or marijuana), who have lost their memory. These supplements also reduce the rate of recidivism and improve both the nutritional and general well being of the addict. Once craving is diminished and the individual is able to function, he or she is less socially disruptive and can become a productive citizen. In contrast, traditional methods using addictive chemicals may invoke more relapses, create a risk of overdose and are more expensive.

The present inventor previously employed a composition and method for nutritional supplement for drug abuse rehabilitation and a protocol for treatment with a combination of specific supplements for specific addictions using a particular mix of supplements based on the phase of treatment, i.e., detoxification, craving and maintenance. Her invention adds the component of fish oil to aid in neuro-conduction and replaces the supplement Bio-Ester with Bio-GLA to aid in neuro-transmission.

Bio-Ester is Vitamin C which is part of nutrients that aids digestion. It is not particularly useful in conduction of messages in the brain and it can be found in combination with other vitamins even if the particular Bio Ester is not used. Bio-GLA is known as Gamma Linoleic Acid which is an essential fatty acid that is required in aiding electrical conduction (neuro-transmission). It is abundant in the brain and must be present in the body adequately for normal brain function. The addition of fish oil for neuro-conduction and Bio-GLA for neuro-transmission yields synergistic results.

### SUMMARY OF THE INVENTION

It is therefore, an object of the present invention to provide a natural alternative to prescription drug treatment for detoxification and medical craving management of drug addicts.

It is the object of the present invention to provide a nutritional supplement and methods of use thereof for drug abuse treatment and rehabilitation.

It is another object of the present invention to provide a nutritional supplement and methods of use thereof for drug abuse treatment and rehabilitation that combines nutritional supplements based on the type of addiction.

It is yet another object of the present invention to provide a nutritional supplement and methods of use thereof for drug abuse treatment and rehabilitation that combines nutritional supplements based on the phase of recovery, i.e., detoxification, craving and maintenance.

It is still another object of the present invention to provide a nutritional supplement protocol comprising effective amounts of Bio-Amino, Biovitamins, Ca & Mg, Vitamin B6, Bio-GLA, L-Glutamine, Tyrosine, and Endorphynyl.

According to the present invention, these and other objects are accomplished by providing a treatment protocol of general supplements: Bio-Amino, Biovitamins, Calcium and Magnesium, and Vitamin B6, plus a drug-specific constituent comprising any one from among the group of Bio-GLA, L-Glutamine, Tyrosine, and Endorphenyl, the selction depending on the addiction being treated. The foregoing are administered in varying combinations and dosages depending on the phase of treatment, i.e., detoxification, craving and maintenance. The individual combinations work to improve neural conduction, particularly in those subjects with severe addiction (particularly addition to heroin or marijuana), who have lost their memory. These protocols also reduce the rate of recidivism and improve both the nutritional and general well being of the addict.

### BRIEF DESCRIPTION OF THE DRAWINGS

Other objects, features, and advantages of the present invention will become more apparent from the following detailed description of the preferred embodiment and certain modifications thereof when taken together with the accompanying drawings in which:

FIG. 1 is a diagram of the limbic system.

FIG. 2 (A through H) are a composite of schematic diagrams that show how common substance abuse affects the limbic system and how they may induce Euphoria.

FIG. 3 is a diagram of the three phases of treatment of the present invention.

### DETAILED DESCRIPTION OF THE PREFERRED EMBODIMENTS

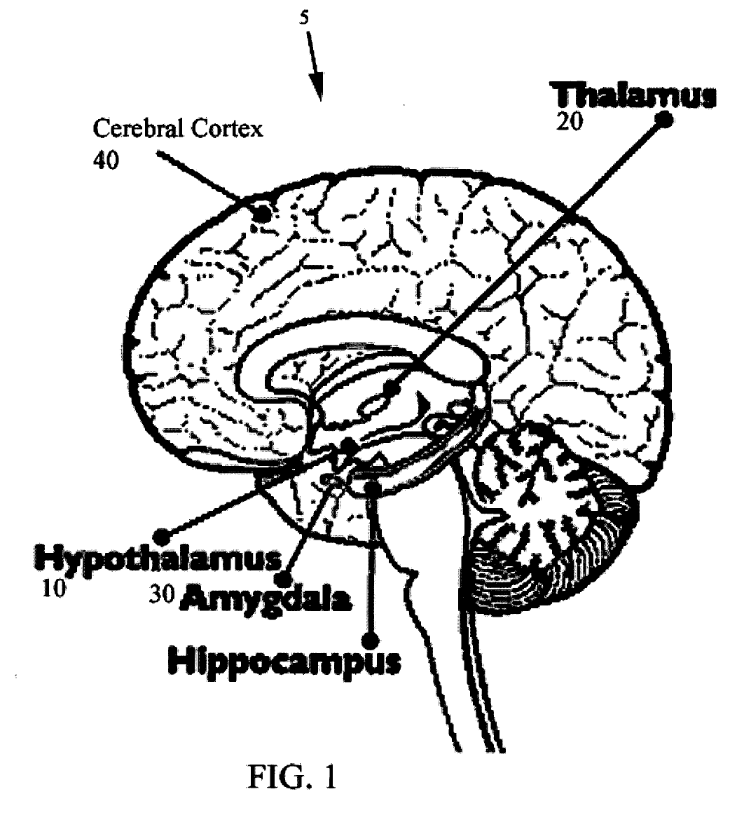

Abusive use of drugs affects the limbic system of the user. FIG. 1 is a diagram of the limbic system, a complex group of nuclei and tracts present in the mid-brain. The limbic system 5 includes, among other things, the hypothalamus 10, thalamus 20, amygdale 30, and part of the cerebral cortex 40. The hypothalamus 10 controls memory and the drive for hunger, sex, and aggression. The thalamus 20 serves as a relay center for pain sensations. The amygdale 30 controls motivation and aggression. The cerebral cortex 40 controls motor coordination. The system integrates emotional and motivational behavior, particularly motor coordination in emotional responses. Neurotransmitters are naturally occurring bio-chemicals that transmit information from one neuron to another in the brain. Receptors bind neurotransmitters before they can transmit information from one neuron to another, allowing an individual to function. Substances of abuse can bind their receptors and displace the neurotransmitters, thereby adversely affecting the individual's limbic system and thus some of their brain function, such as memory, aggression and motor coordination. Nutritional supplements may replenish that which these illicit agents have displaced or diminished.

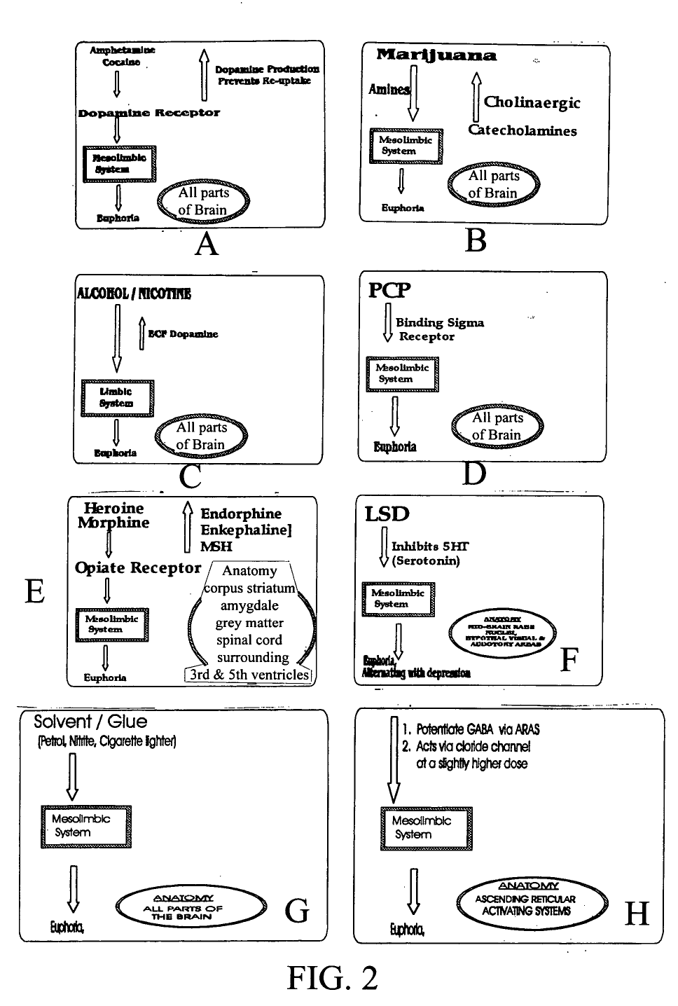

FIGS. 2A through 2H are schematic diagrams that show how common substance abuse affects the limbic system and how illicit drugs may induce Euphoria. FIG. 2A illustrates cocaine induced euphoria. Cocaine, a stimulant, affects all parts of the brain, particularly the mesolimbic system. Cocaine binds at the dopamine receptors and stimulates dopamine production. When an overabundance of dopamine is produced, neuro-transmitter re-uptake is inhibited, which induces a feeling of euphoria. FIG. 2B illustrates marijuana induced euphoria. Marijuana, a hallucinogen, affects all parts of the brain, particularly the mesolimbic system. Marijuana impacts the amines, particularly the catecholamines and cholinaergics, creating a feeling of euphoria. FIG. 2C illustrates alcohol induced euphoria. Alcohol, a depressant, affects all parts of the brain, particularly the limbic system. Alcohol increases the ECF (ExtraCellular Fluid), creating an overabundance, and hence a feeling of euphoria is induced. FIG. 2D illustrates PCP induced euphoria. PCP, a hallucinogen, affects all parts of the brain, particularly the mesolimbic system. PCP binds at the Sigma receptors, creating a feeling of euphoria. FIG. 2E illustrates heroine/morphine induced euphoria. Heroin and morphine, which are opiates, affect the mesolimbic system, particularly the amygdala, corpus stratum, gray matter, and the spinal cord surrounding the 3^{rd} and 4^{th} ventricles. Heroin and morphine bind at the opiate receptors, releasing endorphins, enkephalines, and MSH, inducing a feeling of euphoria FIG. 2F illustrates LSD induced euphoria. LSD, a hallucinogen, affects the mesolimbic system, particularly the mid-brain and the hypothalamus visual and auditory areas. LSD inhibits serotonin production, which lead to feelings of euphoria alternating with depression. FIG. 2G illustrates solvent/glue-induced euphoria. Solvents, such as petrol and nitrite, and glue, which are depressants affect all parts of the brain, particularly the mesolimbic system, and induce a feeling of euphoria. FIG. 2H illustrates Valium (tm)/barbiturate induced euphoria. Valium (tm) and barbiturates, which are depressants, affect the ascending reticular activating systems of the mesolimbic system, creating a feeling of euphoria.

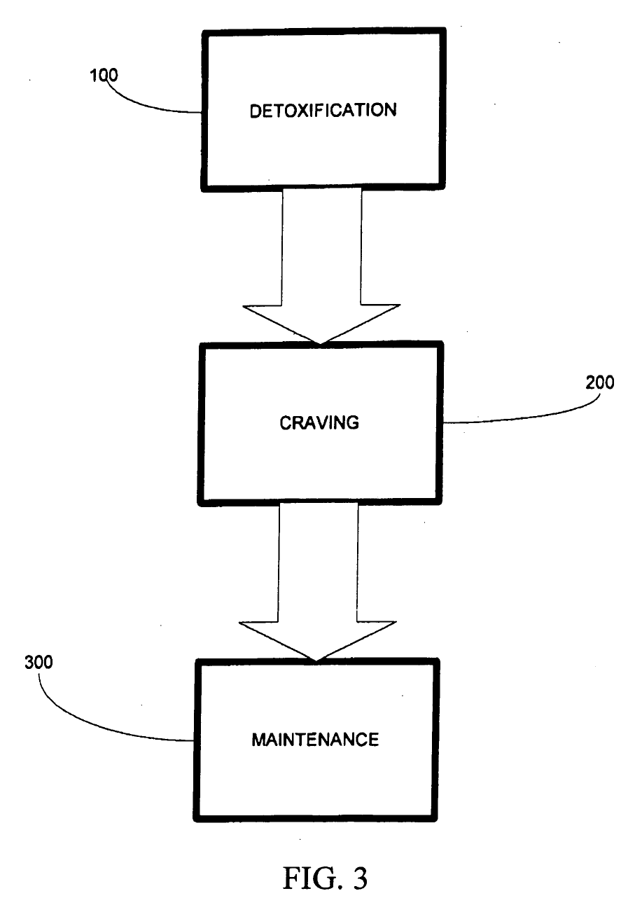

FIG. 3 is a diagram of the three phases of treatment of the present invention. Nutritional supplements have been shown to significantly relieve craving, depression and exhaustion, which lead addicts to relapse following detoxification. Nutritional supplements are basic amino acids and proteins that act as building blocks for the body cells, tissues and chemical substances. The brain needs these supplements to produce the neurotransmitters, which are needed for basic bodily functions and emotional responses. Step 100 is the Detoxification phase. Detoxification is the process through which a person who is physically dependent on alcohol, illegal drugs, prescription drugs, or a combination of these drugs is withdrawn from the drugs of dependence. Detoxification implies clearing the user's system of toxins, but also includes the time during which the user's physiology is adjusting to the absence of drugs. It is a series of processes to alleviate short-term symptoms of withdrawal from drug dependence. Sudden removal of alcohol or another drug of abuse from the system of a physically dependent user may produce withdrawal syndrome. The immediate goal of detoxification is a safe withdrawal from the addictive drug of dependence to enable the user to become drug-free. The signs and symptoms of withdrawal are usually the reverse of the direct pharmacological effects of the drug. For example, heroin use commonly produces euphoria, a decrease in anxiety and insensitivity to pain, while withdrawal from heroin, on the other hand, produces an unpleasant mood, pain and anxiety. There are a number of risks associated with withdrawal, such as physical discomfort and pain, delirium, and other life-threatening symptoms. A secondary goal of detoxification is to prepare the addict for appropriate follow up treatment. Traditional detoxification treatment involves administering detoxification medication to the user in lieu of the user's addictive drug. Some detoxification procedures are specific to particular addictive drugs, while others are not drug-specific. Medical detoxification is a process whereby the addict is systematically withdrawn from the addictive drug, typically under the care of a physician. Detoxification can be considered a precursor for long-term treatment because it is designed to treat the acute physiological effects that occur when the user stops taking his or her addictive drug. Traditionally, medications are available for detoxification. In some cases, detoxification may be a medical necessity, and untreated withdrawal may be medically dangerous, or in extreme cases, fatal. Professional treatment of withdrawal during detoxification is critical to the health and welfare of the user. The present invention is a protocol that utilizes a combination of general and drug-specific nutritional supplements. The detoxification protocol of the present invention is a two-week protocol as follows. The last row of the table shows the additional supplement specific to the type of drug addiction, while all other rows show general supplements.

| Alcohol | Cocaine | Heroin | Marijuana |
|---|---|---|---|
| Bio-Amino Caps | Bio-Amino Caps | Bio-Amino Caps | Bio-Amino Caps |
| 2.8 G stat (4 caps); then | 2.8 G stat (4 caps); then | 2.8 G stat (4 caps); then | 2.8 G stat (4 caps); then |
| 1.4 G (2 caps) tid/qid | 1.4 G (2 caps) tid/qid | 1.4 G (2 caps) tid/qid | 1.4 G (2 caps) tid/qid |
| Biovitamins 2 caps | Biovitamins 2 caps | Biovitamins 2 caps | Biovitamins 2 caps |
| | tid/qid | tid/qid | tid/qid |
| Ca & Mg 200 mg (2 | Ca & Mg 200 mg (2 | Ca & Mg 200 mg (2 | Ca & Mg 200 mg (2 |
| caps) tid/qid | caps) tid/qid | caps) tid/qid | caps) tid/qid |
| Vitamin B6 250 mg (1 | Vitamin B6 250 mg (1 | Vitamin B6 250 mg (1 | Vitamin B6 250 mg (1 |
| tab or cap) tid/qid | tab or cap) tid/qid | tab or cap) tid/qid | tab or cap) tid/qid |
| Bio-GLA 1300 mg (1 | Bio-GLA 1300 mg (1 | Bio-GLA 1300 mg (1 | Bio-GLA 1300 mg (1 |
| gel) tid/qid | gel) tid/qid | gel) tid/qid | gel) tid/qid |
| L-Glutamine 500 mg | Tyrosine 1600 mg (2 | Endorphenyl 1000 mg | Bio-GLA 1300 mg (1 |
| (2 caps) tid/qid | caps) tid/qid | (2 caps) tid/qid | gel) tid/qid |

Step 200 is the Craving phase. When a user continuously uses drugs, an overabundance of metabolites is present and stored in the user's body, sometimes for years. When the user tries to quit using the addictive drug, the drug metabolites can be released back into the blood stream and reactivate the same brain centers as if the person actually just took the drug. The former addict experiences a drug restimulation and drug craving, which are almost impossible to resist. Presence of these metabolites in the blood cause the brain to react as if the addict were withdrawing from the drug. Receptors in the brain that have adapted to large amounts of the drug metabolite now must deal with only a small amount of metabolite. The brain asks the addict for more of the drug. This is known as drug craving. The only way to end craving is to take more drugs and the cycle continues. Traditional treatment for cravings includes administering medication to eliminate the drug cravings. The protocol of the present invention uses a combination of general and drug-specific nutritional supplements, in lieu of the traditional treatment of prescription drugs. The craving protocol of the present invention is a six-week protocol that varies depending upon the severity of the craving, shown in the following tables.

The last row of the table shows the additional supplement specific to the type of drug addiction, while all other rows show general supplements.

Severe Craving:

| Alcohol | Cocaine | Heroin | Marijuana |
|---|---|---|---|
| Bio-Amino Caps | Bio-Amino Caps | Bio-Amino Caps | Bio-Amino Caps |
| 1.4 G stat (2 caps) | 1.4 G stat (2 caps) | 1.4 G stat (2 caps) | 1.4 G stat (2 c tid/qid |
| tid/qid | tid/qid | tid/qid | aps) |
| Biovitamins 2 caps | Biovitamins 2 caps | Biovitamins 2 caps | Biovitamins 2 caps |
| Ca & Mg 200 mg (2 | Ca & Mg 200 mg (2 | Ca & Mg 200 mg (2 | Ca & Mg 200 mg (2 |
| caps) tid/qid | caps) tid/qid | caps) tid/qid | caps) tid/qid |
| Vitamin B6 250 mg (1 | Vitamin B6 250 mg (1 | Vitamin B6 250 mg (1 | Vitamin B6 250 mg (1 |
| tab or cap) tid/qid | tab or cap) tid/qid | tab or cap) tid/qid | tab or cap) tid/qid |
| Bio-GLA 1300 mg (1 | Bio-GLA 1300 mg (1 | Bio-GLA 1300 mg (1 | Bio-GLA 1300 mg (1 |
| gel) tid/qid | gel) tid/qid | gel) tid/qid | gel) tid/qid |
| L-Glutamine 1000 mg | Tyrosine 1600 mg (2 | Endorphenyl 1000 mg | Bio-GLA 1300 mg (1 |
| (2 caps) | caps) tid/qid | (2 caps) tid/qid | gel) tid/qid |

Mild/Moderate Craving:

| Alcohol | Cocaine | Heroin | Marijuana |
|---|---|---|---|
| Bio-Amino Caps | Bio-Amino Caps | Bio-Amino Caps | Bio-Amino Caps |
| 700 mg (1 cap) tid/qid | 700 mg (1 cap) tid/qid | 700 mg (1 cap) tid/qid | 700 mg (1 cap) tid/qid |
| Biovitamins 1 cap | Biovitamins 1 cap | Biovitamins 1 | Biovitamins 1 cap |
| tid/qid | tid/qid | cap tid/qid | tid/qid |
| Ca & Mg 200 mg (2 | Ca & Mg 200 mg (2 | Ca & Mg 200 mg (2 | Ca & Mg 200 mg (2 |
| caps) tid/qid | caps) | caps) tid/qid | caps) tid/qid |
| Vitamin B6 250 mg (1 | Vitamin B6 250 mg (1 | Vitamin B6 250 mg (1 | Vitamin B6 250 mg (1 |
| tab or cap) tid/qid | tab or cap) tid/qid | tab or cap) tid/qid | tab or cap) tid/qid |
| Bio-GLA 1300 mg (1 | Bio-GLA 1300 mg (1 | Bio-GLA 1300 mg (1 | Bio-GLA 1300 mg (1 |
| gel) tid/qid | gel) tid/qid | gel) tid/qid | gel) tid/qid |
| L-Glutamine 500 mg | Tyrosine 800 mg (1 | Endorphenyl 500 mg | Bio-GLA 1300 mg (1 |
| (1 cap) tid/qid | cap) tid/qid | (1 cap) tid/qid | gel) tid/qid |

Step 300 is the Maintenance phase, which is a major step to long term recovery. Detoxification is not designed to address the psychological, social and behavioral problem associated with addiction and thus does not usually produce lasting behavioral changes necessary for recovery. The maintenance phase addresses the period after detoxification and craving, when the former addict is drug-free. The maintenance protocol of the present invention is a six month to one year protocol as follows. The last row of the table shows the additional supplement specific to the type of drug addiction, while the first row show general supplements.

| Alcohol | Cocaine | Heroin | Marijuana |
|---|---|---|---|
| Biovitamins 1 cap | Biovitamins 1 cap | Biovitamins 1 cap | Biovitamins 1 cap |
| Vitamin B6 250 mg (1 | Vitamin B6 250 mg (1 | Vitamin B6 250 mg (1 | Vitamin B6 250 mg (1 |
| tab or cap) | tab or cap) | tab or cap) | tab or cap) |
| Bio-GLA 1300 mg (1 | Bio-GLA 1300 mg (1 | Bio-GLA 1300 mg (1 | Bio-GLA 1300 mg (1 |
| gel) | gel) | gel) | gel) |
| L-Glutamine 500 mg | Tyrosine 800 mg (1 | Endorphenyl 500 mg | Bio-GLA 1300 mg (1 |
| (1 cap) | cap) | (1 cap) | gel) |

The foregoing nutritional supplement and regimen of administration improve the efficacy of drug abuse in three distinct phases: detoxification, medical craving management and rehabilitation.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

1. A combination of nutritional supplements for drug abuse treatment and rehabilitation, comprising a mixture of:
(a) Bio-Aminos;
(b) Biovitamins;
(c) Calcium;
(d) Magnesium;
(e) Vitamin B6; and
(f) a drug-specific constituent comprising any one from among the group of Bio-GLA, L-Glutamine, Tyrosine, and Endorphenyl.

2. The combination of claim 1 for detoxification of alcohol, wherein said drug-specific constituent is L-Glutamine.

3. The combination of claim 1 for detoxification of cocaine, wherein said drug-specific constituent is Tyrosine.

4. The combination of claim 1 for detoxification of heroin, wherein said drug-specific constituent is endorphenyl.

5. The combination of claim 1 for detoxification of marijuana, wherein said drug-specific constituent is Bio-GLA.

6. A method for drug abuse detoxification, medical craving management and rehabilitation from cocaine, heroin, alcohol, or marijuana addiction, comprising administering a regimen of nutritional supplements over a time interval, said nutritional supplements including a combination of general supplements and a specific supplement, said specific supplement being determined in accordance with addiction-type whether cocaine, heroin, alcohol, or marijuana, and said specific supplement being administered in varying quantity over said time interval beginning with a first quantity given during an initial detoxification phase, changing to a second quantity given during a craving phase, and ending with a third quantity given during a maintenance phase.

7. The method of claim 6 wherein the general supplements include Bio-Aminos, Biovitamins, Calcium, Magnesium and Vitamin B6.

8. The method of claim 7 wherein the drug-specific supplement comprises any one from among the group of Bio-GLA, L-Glutamine, Tyrosine, and Endorphenyl.

9. The method of claim 8 only for alcohol addiction, wherein said drug-specific supplement is L-Glutamine.

10. The method of claim 8 only for cocaine addiction, wherein said drug-specific supplement is Tyrosine.

11. The method of claim 8 only for heroin addiction, wherein said drug-specific supplement is Endorphenyl.

11. The method of claim 8 only for marijuana addiction, wherein said drug-specific supplement is Bio-GLA.

12. The method of claim 7 wherein the combination of nutritional supplements is administered over said time interval in varying amounts beginning with a first quantity given during an initial detoxification phase lasting two weeks, changing to a second quantity given during a craving phase lasting six weeks, and ending with a third quantity given during a maintenance phase lasting between six months to one year.

13. A combination of nutritional supplements for treatment and rehabilitation from any one of alcohol, cocaine, marijuana or heroin, comprising a general mixture of Bio-Aminos, Biovitamins, Calcium, Magnesium and Vitamin B6, and a drug-specific constituent chosen from among a group consisting of Bio-GLA, L-Glutamine, Tyrosine, and Endorphenyl, depending on whether said combination will be used to treat an alcohol, cocaine, marijuana or heroin addiction.

14. The combination of claim 13 for alcohol addiction, wherein said drug-specific supplement is L-Glutamine.

15. The combination of claim 13 for cocaine addiction, wherein said drug-specific supplement is Tyrosine.

16. The combination of claim 13 for heroin addiction, wherein said drug-specific supplement is Endorphenyl.

17. The combination of claim 13 for marijuana addiction, wherein said drug-specific supplement is Bio-GLA.
